(19) United States
(12) Patent Application Publication
(10) Pub. No.: US 2019/0217078 A1
(43) Pub. Date: Jul. 18, 2019

(54) ELECTRODE

(71) Applicant: ETEXSENSE LIMITED, SOUTHAMPTON (GB)

(72) Inventors: KAI YANG, SOUTHAMPTON (GB); CHRISTOPHER FREEMAN, SOUTHAMPTON (GB)

(21) Appl. No.: 16/302,902

(22) PCT Filed: May 17, 2017
(86) PCT No.: PCT/GB2017/051377

§ 371 Date: Nov. 19, 2018

(30) Foreign Application Priority Data
May 17, 2016 (GB) 1608691.0

(51) Int. Cl. A61N 1/04; A61B 5/0408; A61B 5/0478; A61B 5/0492; A61B 5/00

(52) U.S. Cl. CPC A61N 1/0496; A61B 5/0408; A61B 5/0478; A61B 5/0492; A61B 2562/125; A61N 1/0452; A61N 1/0456; A61N 1/0484; A61B 5/6802

(57) ABSTRACT

A wearable dry electrode comprising: a conductive layer; and a contact layer electrically connected to the conductive layer; wherein the contact layer comprises carbon and rubber.

### FIELD OF THE INVENTION

The invention relates to an electrode, in particular, but not exclusively, an electrode for an at least partially wearable device. More particularly, the invention relates to a wearable substantially dry electrode, and particularly to a wearable electrode suitable for applying a current into the skin of a wearer, or detecting a biopotential in the skin of a wearer. The invention further relates to garments comprising such electrodes, methods of manufacture of such electrodes and methods of manufacture of garments comprising such electrodes.

### BACKGROUND

Wearable technology is a rapidly expanding area of technology, offering a multitude of opportunities. For example, wearable medical devices can be used for disease treatment, therapy, rehabilitation, health monitoring and assisting home-based independent living. Thus, an increased use of wearable medical devices could provide efficient and/or effective disease treatment, therapy, rehabilitation and/or health monitoring, while reducing cost and staffing burdens on health services or healthcare providers. Another example of wearable technology is wearable fitness devices. A wearable fitness device may be operable to monitor a wearer's physical condition or performance, e.g. during exercise. Another type of wearable fitness device may be operable to provide muscle stimulation.

Electrodes play an extremely important role as a fundamental element in such devices, e.g. medical devices, healthcare devices or fitness devices. Electrodes can provide an electrical contact with the skin, allowing electrical measurements of the wearer, or allowing small electrical currents to be applied to the wearer. It has been estimated that the market for medical electrodes alone will be worth around $800 million per year by 2025.

Conventional electrodes in wearable devices fall into two categories, wet/hydrogel electrodes; and dry electrodes. Wet electrodes require skin preparation, such as applying a liquid gel on the skin, whereas hydrogel electrodes comprise a layer of hydrogel to enhance the contact between the skin and the electrode. However, neither of these electrodes are suitable for long term use—applying the liquid gel to the skin can be time consuming, and the electrode cannot be reused many times due to likely contamination; and hydrogel electrodes tend to dry out. Furthermore, the high degree of skin adhesion makes hydrogel electrodes difficult to use. For example it can be hard to reposition the electrodes due to their stickiness, which can be painful for the wearer.

Conventional dry electrodes tend to be fabricated using materials which have a poor contact with the skin. This can cause an uneven distribution of current which may lead to discomfort or even pain for the wearer. Therefore, it tends to be necessary to apply a gel layer either on the skin or on the electrode. This is not convenient for long term wearable applications.

US2015/0202429 discloses a device for muscle stimulation. The device comprises a dry electrode integrated into a garment for muscle stimulation for sports and medical rehabilitation.

U.S. Pat. No. 8,406,841 discloses a dry electrode for a biomedical signal measuring sensor. The dry electrode comprises a conductive sponge, a conductive fabric and a thin metal film.

“Screen printed fabric electrode array for wearable functional electrical stimulation”, Sensors and Actuators A: Physical. 213, pp. 108-115, 2014 discloses a fully printed fabric electrode array for muscle stimulation. The electrode array comprises four functional layers—an interface layer, a conductive layer, an encapsulation layer and a contact layer—which were screen printed.

“Textile Neuroprosthesis Garment for Functional Electrical Stimulation”, International Workshop on Functional Electrical Stimulation. Krems. Austria, no. 9. pp. 107-110 discloses a textile neuroprosthesis garment, in which conductive tracks and electrode pads are fabricated using embroidery.

“Parylene-based flexible dry electrode for biopotential recording”, Sensors and Actuators B: Chemical, Available online 17 Feb. 2016. doi:10.1016/j.snb.2016.02.061 discloses a dry electrode with thousands of AgCl micropads for biopotential recording.

### SUMMARY OF THE INVENTION

In accordance with a first aspect of the invention there is provided an electrode comprising: a conductive layer; and a contact layer electrically connected to the conductive layer: wherein the contact layer comprises carbon and a rubber.

The electrode may be an at least partially wearable, substantially dry electrode, e.g. a wearable dry electrode.

The contact layer may overlay at least a portion of the conductive layer.

In some embodiments, the conductive layer may comprise one or more conductive elements or regions. Typically, the electrode may be configured such that, in use, no electrically conductive parts of the conductive layer can come into contact with a user's skin. For example, the contact layer may overlay all of the electrically conductive parts of the conductive layer.

The electrode may comprise two or more functional layers. Typically, the electrode may comprise only two functional layers, i.e. the conductive layer and the contact layer. In other embodiments, the electrode may comprise more than two functional layers. For instance, the electrode may comprise an insulating layer on the opposite side of the conductive layer from the contact layer, i.e. the conductive layer may be located between the insulating layer and the contact layer.

The contact layer may comprise an ink or paste, e.g. a cured ink or paste, the ink or paste comprising a mixture containing carbon and the rubber.

Advantageously, the contact layer may increase the contact between the skin and the electrode, allowing accurate measurements of the (human or animal) wearer to be taken, for example by a wearable medical device attached to the electrode. Conveniently, the electrode may not require a gel to be applied to the skin, and so may be more suitable for long-term use than a conventional electrode. In particular, such an electrode may be relatively easy to locate on the skin, and substantially painless to remove from the skin.

By weight, the contact layer or ink or paste may comprise at least 5 parts carbon per 100 parts rubber and/or up to 40 parts carbon per 100 parts rubber. The contact layer or ink or paste may comprise up to or at least 10 parts carbon per 100 parts rubber and/or up to or at least 33 parts carbon per 100 parts rubber. For example, the contact layer or ink or paste may comprise up to or at least 12 parts carbon per 100 parts rubber or up to or at least 18 parts carbon per 100 parts rubber. For instance, the contact layer or ink or paste may comprise approximately 15 parts carbon per 100 parts rubber. In other embodiments, the contact layer or ink or paste may for example comprise up to or at least 20 parts carbon per 100 parts rubber or up to or at least 25 parts carbon per 100 parts rubber. For example, the contact layer or ink or paste may comprise approximately 22.5 parts carbon per 100 parts rubber. In other embodiments, the contact layer or ink or paste may for example comprise up to or at least 27 and/or up to 33 parts carbon per 100 parts rubber. For instance, the contact layer or ink or paste may comprise approximately 30 parts carbon per 100 parts rubber.

It has been found that contact layers comprising such proportions of carbon to rubber may provide sufficient electrical contact between the skin and the contact layer, without requiring an extra skin interface layer or agent, e.g. a gel, to enhance the contact between the skin and electrode.

In some embodiments, the contact layer or ink or paste may further comprise a modifier for modifying the tackiness of the rubber. Accordingly, the tackiness of the contact layer may be modified and/or controlled. Advantageously, a tacky contact layer may provide an effective and/or secure contact between the electrode and a user's skin. By weight, the contact layer or ink or paste may for example comprise up to or at least 20 parts modifier per 100 parts rubber, up to or at least 30 parts modifier per 100 parts rubber, up to or at least 45 parts modifier per 100 parts rubber and/or up to 110 parts modifier per 100 parts rubber. The contact layer or ink or paste may comprise up to or at least 45 parts modifier per 100 parts rubber, up to or at least 55 parts modifier per 100 parts rubber, up to or at least 95 parts modifier per 100 parts rubber, or up to or at least 105 parts per modifier per 100 parts rubber. Increasing the amount of modifier in the contact layer or ink or paste may increase the tackiness of the rubber.

Typically, the rubber may comprise a silicone rubber.

In embodiments comprising silicone rubber, the optional modifier may comprise a silicone modifier for modifying the tackiness of the silicone rubber. The silicone modifier may for example comprise Slacker® produced by Smooth-On, Inc.

It has been found that increasing the tackiness of the contact layer by adding a modifier, e.g. a silicone modifier, can enhance the contact between the skin and the electrode, without making the electrode too sticky. However, increased amounts of modifier may make the ink more difficult to cure.

In some embodiments, the contact layer, ink or paste may further comprise a thinner solvent. For example the thinner solvent may be a butyl carbitol acetate-based thinner, for example ESL T-402 produced by ESL Electroscience. The thinner solvent may make the ink less viscous, so that it may be more easily handled and/or applied when producing or forming the contact layer.

In some embodiments, the contact layer, ink or paste may further comprise a metal or other electrically conductive material, e.g. silver or copper.

In an embodiment, the carbon may comprise graphene.

In some embodiments, the contact layer may be produced by depositing, e.g. coating or printing, the or an ink or paste comprising a mixture containing carbon and a rubber, e.g. a silicone rubber onto the conductive layer. For example, the ink or paste may be stencil printed onto the conductive layer.

In other embodiments, the contact layer may be provided as a pre-formed piece. The pre-formed piece may be attached to the conductive layer by any suitable means. Conveniently, the pre-formed piece may be attached to the conductive layer by an adhesive, e.g. a glue, and/or a mechanical fixing means, e.g. by sewing.

In an embodiment, the conductive layer may comprise at least one conductive yarn or conductive wire integrated into a textile or fabric. For example, the conductive yarn(s) or wire(s) may be woven, embroidered, knitted or sewn into the textile or fabric, or otherwise fixed, bonded or adhered onto the textile or fabric. The conductive yarn(s) or wire(s) may be arranged in any pattern. e.g. a serpentine pattern, a spiral pattern or a grid-like pattern.

The textile or fabric may be integrated into a garment for a human or an animal. For example, the garment may comprise an arm band, chest band, hat, head band, sock, leg band, sleeve, t-shirt, shirt, leggings, tights, shorts or body suit. The textile or fabric may be integrated into the garment by any suitable means, e.g. using sewing, adhesive, or hook and loop fasteners.

The textile or garment may be an at least partially elasticated or stretchable textile or garment. Such embodiments may provide an electrode with close contact to the skin, whilst maintaining comfort for the wearer.

The conductive yarn(s) or conductive wire(s) may each comprise, for example, a metallic fibre or a metal-coated fibre, e.g. a stainless steel fibre, an aluminium or aluminium alloy fibre, a copper fibre, or a silver-coated fibre.

In some embodiments, an external connector, such as a connector for connecting the electrode to a device, for example a fitness device, a healthcare device or a medical device, may be electrically connected to the conductive layer. For example, the external connector may be electrically connected to the conductive yarn(s) or conductive wire(s) in the conductive layer. The external connector may for example be a pigtail connector.

In an embodiment, the electrode may be flexible at least in part.

In an embodiment, at least a portion of the surface of the contact layer for contacting, in use, a user's skin may be rough or contoured, e.g. may comprise one or more grooves, projections or nodules. At least a portion of the surface of the contact layer for contacting, in use, a user's skin may be curved, e.g. concave or convex. Providing a rough, contoured and/or curved surface or portion thereof of the contact layer may assist in providing a better contact with a user's skin, particularly, but not exclusively if the user's skin is relatively hairy.

According to a second aspect of the invention there is provided a garment comprising: a fabric; and at least one electrode according to any embodiment of the first aspect of the invention attached to the fabric.

The electrode may be integrated into the fabric, for example the electrode may be interwoven or sewn into the fabric. Alternatively, the electrode may be bonded to the fabric, e.g. adhered to the fabric using an adhesive.

In particular embodiments, the fabric may comprise, or consist essentially of, an at least partially elasticated or stretchable fabric.

The garment may be designed to be worn by a human or an animal. For example, the garment may comprise an arm band, chest band, hat, head band, sock, leg band, sleeve, t-shirt, shirt, leggings, tights shorts or body suit.

According to a third aspect of the invention there is provided an ink or paste for forming a contact layer of an electrode, the ink or paste comprising a mixture of carbon and a rubber.

Typically, the ink or paste may be curable.

The ink or paste may be suitable for depositing, e.g. by coating, printing or pasting, in use, onto a conductive layer of a wearable substantially dry electrode.

By weight, the ink or paste may comprise at least 5 parts carbon per 100 parts rubber and/or up to 40 parts carbon per 100 parts rubber. The contact layer or ink or paste may comprise up to or at least 10 parts carbon per 100 parts rubber and/or up to or at least 33 parts carbon per 100 parts rubber. For example, the ink may comprise up to or at least 12 parts carbon per 100 parts rubber or up to or at least 18 parts carbon per 100 parts rubber. For instance, the ink may comprise approximately 15 parts carbon per 100 parts rubber. In other embodiments, the ink or paste may for example comprise up to or at least 20 parts carbon per 100 parts rubber or up to or at least 25 parts carbon per 100 parts rubber. For example, the ink or paste may comprise approximately 22.5 parts carbon per 100 parts rubber. In other embodiments, the ink or paste may for example comprise up to or at least 27 and/or up to 33 parts carbon per 100 parts rubber. For instance, the ink or paste may comprise approximately 30 parts carbon per 100 parts rubber.

In some embodiments, the ink or paste may further comprise a modifier for modifying the tackiness of the rubber. By weight, the ink or paste may for example comprise up to or at least 20 parts modifier per 100 parts rubber, up to or at least 30 parts modifier per 100 parts rubber, up to or at least 45 parts modifier per 100 parts rubber and/or up to 110 parts modifier per 100 parts rubber. The ink or paste may comprise up to or at least 45 parts modifier per 100 parts rubber, up to or at least 55 parts modifier per 100 parts rubber, up to or at least 95 parts modifier per 100 parts rubber, or up to or at least 105 parts per modifier per 100 parts rubber.

Typically, the rubber may comprise a silicone rubber.

In embodiments comprising silicone rubber, the optional modifier may comprise a silicone modifier for modifying the tackiness of the silicone rubber. The silicone modifier may for example comprise Slacker® produced by Smooth-On, Inc.

In some embodiments, the ink or paste may further comprise a thinner solvent. For example the thinner solvent may be a butyl carbitol acetate-based thinner, for example ESL T-402 produced by ESL Electroscience. The thinner solvent may make the ink or paste less viscous, so that it may be more easily handled and/or applied when producing or forming the contact layer.

In some embodiments, the contact layer, ink or paste may further comprise a metal or other electrically conductive material, e.g. silver or copper.

In an embodiment, the carbon may comprise graphene.

According to a fourth aspect of the invention there is provided an at least partially wearable device comprising an electrode according to any embodiment of the first aspect of the invention. For example, the device may comprise a fitness device, a healthcare device or a medical device.

According to a fifth aspect of the invention there is provided a garment comprising an electrode according to any embodiment of the first aspect of the invention.

According to a sixth aspect of the invention there is provided a method of producing an electrode, the method comprising: providing a conductive layer: and arranging a contact layer comprising carbon and a rubber in electrical connection with the conductive layer.

Typically, the electrode may comprise only two functional layers, i.e. the conductive layer and the contact layer.

The contact layer may overlay at least a portion of the conductive layer.

In some embodiments, the conductive layer may comprise one or more conductive elements or regions. Typically, the electrode may be configured such that, in use, no electrically conductive parts of the conductive layer can come into contact with a user's skin. For example, the contact layer may overlay all of the electrically conductive parts of the conductive layer.

In some embodiments, the contact layer may be provided as a preformed piece. Conveniently, the preformed piece may be cut from a larger, preformed sheet.

The preformed piece may be attached, e.g. bonded or adhered and/or sewn, to the conductive layer.

In an embodiment, the method may comprise the preliminary step of forming a preformed piece or a larger, preformed sheet from which the preformed piece can be cut, the preformed piece subsequently providing the contact layer. Forming the preformed piece of the preformed sheet may comprise: arranging an ink or paste comprising a mixture of carbon and the rubber in a desired form for the preformed piece or preformed sheet; and, optionally, curing the ink or paste.

In an embodiment, the contact layer may be formed by depositing, e.g. coating, printing or pasting, an ink or paste onto the conductive layer, the ink or paste comprising a mixture of carbon and the rubber; and, optionally, curing the ink or paste.

By weight, the contact layer may comprise at least 5 parts carbon per 100 parts rubber and/or up to 40 parts carbon per 100 parts rubber. The contact layer may comprise up to or at least 10 parts carbon per 100 parts rubber and/or up to or at least 33 pans carbon per 100 parts rubber. For example, the contact layer may comprise up to or at least 12 parts carbon per 100 parts rubber or up to or at least 18 parts carbon per 100 parts rubber. For instance, the contact layer may comprise approximately 15 parts carbon per 100 parts rubber. In other embodiments, the contact layer may for example comprise up to or at least 20 parts carbon per 100 parts rubber or up to or at least 25 parts carbon per 100 parts rubber. For example, the contact layer may comprise approximately 22.5 parts carbon per 100 parts rubber. In other embodiments, the contact layer may for example comprise up to or at least 27 and/or up to 33 parts carbon per 100 parts rubber. For instance, the contact layer may comprise approximately 30 parts carbon per 100 parts rubber.

The contact layer may further comprise a modifier for modifying the tackiness of the rubber.

By weight, the contact layer may comprise up to or at least 20 parts modifier per 100 parts rubber, up to or at least 30 parts modifier per 100 parts rubber, up to or at least 45 parts modifier per 100 parts rubber and/or up to 110 parts modifier per 100 parts rubber. The contact layer may comprise up to or at least 45 parts modifier per 100 parts rubber, up to or at least 55 parts modifier per 100 parts rubber, up to or at least 95 parts modifier per 100 parts rubber, or up to or at least 105 parts per modifier per 100 parts rubber.

Typically, the rubber may comprise a silicone rubber.

In embodiments comprising silicone rubber, the optional modifier may comprise a silicone modifier for modifying the tackiness of the silicone rubber. The silicone modifier may for example comprise Slacker® produced by Smooth-On, Inc.

In some embodiments, the contact layer may further comprise a thinner solvent. For example the thinner solvent may be a butyl carbitol acetate-based thinner, for example ESL T-402 produced by ESL Electroscience. The thinner solvent may make the ink or paste less viscous, so that it may be more easily handled and/or applied when producing or forming the contact layer.

In some embodiments, the contact layer may further comprise a metal or other electrically conductive material, e.g. silver or copper.

In an embodiment, the carbon may comprise graphene.

In an embodiment, the method may comprise the preliminary step of forming the rubber. The rubber, e.g. silicone rubber, may comprise a multi-component system, e.g. a two component system, whereby the components are brought together and react to form the rubber. For instance, the components, e.g. two components, may react together at room temperature to form the rubber. In such an embodiment, the ink or paste may not need to be heated, in order to form the contact layer, preformed piece, or larger, preformed sheet from which a preformed piece can be cut.

Heating the ink or paste to cure the ink or paste may allow for the contact layer, preformed piece or larger, preformed sheet to be formed more quickly. For instance, curing the ink or paste by heating may accelerate the reaction(s) between components of the rubber. In some embodiments, curing the ink or paste may comprise heating the ink or paste to a temperature of up to or at least 40° C., up to or at least 60° C. or up to or at least 70° C. and/or up to 130° C. or up to 150° C. The ink or paste may for example be cured for up to or at least 5 minutes, up to or at least 10 minutes, up to or at least 20 minutes, up to or at least 40 minutes and/or up to or at least 60 minutes.

Even higher curing temperatures may be used, e.g. up to 200° C., in particular in embodiments where the ink or paste is not being cured on the conductive layer. i.e. where the contact layer is being preformed. This is because the conductive layer, which may typically comprise a textile or fabric, may be damaged by curing the ink or paste on the conductive layer at temperatures in excess of 150° C.

In some embodiments, the method may comprise the preliminary step of forming the or an ink or paste by mixing a rubber with carbon, e.g. comprising a conductive carbon powder and/or graphene.

For example, the carbon, e.g. carbon powder and/or graphene, and rubber, e.g. silicone rubber, may be added to give a ratio of at least 5 and/or up to 40 parts carbon per 100 parts rubber in the mixed ink or paste.

Forming the ink or paste may comprise: mixing a modifier for modifying the tackiness of the rubber into the ink or paste, and/or mixing a thinner solvent into the ink or paste.

The conductive layer may comprise one or more conductive elements or regions. The conductive layer may have at least one conductive wire or conductive yarn integrated therein. The conductive yarn(s) or wire(s) may be arranged in any suitable pattern. e.g. a serpentine pattern, a spiral pattern or a grid-like pattern.

In some embodiments, providing a conductive layer may comprise providing a textile or fabric and integrating at least one conductive wire or conductive yarn into the textile or fabric. For example, the conductive yarn(s) or wire(s) may be interwoven, knitted, embroidered or sewn into the textile or fabric, or otherwise fixed, bonded or adhered onto the textile or fabric.

In some embodiments, providing a conductive layer may comprise electrically connecting an external connector to the conductive layer. For example, the external connector may be electrically connected to the conductive wire or yarn. The external connector may comprise a pigtail connector.

Some embodiments may further comprise the step of attaching the electrode to a garment for a human or an animal. For example, the garment may comprise an arm band, chest band, hat, head band, sock, leg band, sleeve, t-shirt, shirt, leggings, tights shorts or body suit. For example, the electrode may be bonded, e.g. adhered, to the garment, sewn, woven knitted or embroidered into the garment, or attached to the garment using hook and loop fasteners.

According to a seventh aspect of the invention there is provided a use of an electrode according to the first aspect of the invention, a garment according to the second aspect of the invention or an at least partially wearable device according to the fourth aspect of the invention to take an electrical measurement. e.g. a biopotential, of a user's skin and/or to apply an electrical current to the user's skin.

The invention may be particularly well suited for use in applying an electrical current to a user's skin, e.g. to provide muscle or nerve stimulation. Such muscle stimulation may provide pain relief and/or muscle exercise, e.g. for chronic pain relief, strengthening pelvic floor muscles, rehabilitation. One example of a suitable application may be for functional electrical stimulation (FES) for stroke rehabilitation, or treatment of other neurological disorders.

By providing a dry electrode having good tackiness, skin contact is improved and consequently user comfort. Consequently, use of the invention may be particularly advantageous for muscle stimulation applications, as muscle stimulation typically requires a relatively high current density (mA/cm²) to be applied to the user's skin, in order to produce activation and movement in the case of rehabilitation. At such relatively high current density levels, use of a normal, dry electrode without a gel layer can lead to the user experiencing discomfort and even pain, due to the relatively poor skin contact. In contrast, the tackiness of the contact layer of the electrode of the present invention provides an improved interface between the electrode and the user's skin, which enables the relatively high current densities required for nerve or muscle stimulation to be applied to the user's skin without causing significant discomfort or pain. The tackiness of the contact layer of the electrode may be determined by the formulation of the ink or paste.

### DETAILED DESCRIPTION

In order that the invention can be well understood, embodiments of the invention will be described in further detail below by way of example only and with reference to the accompanying drawings, in which:

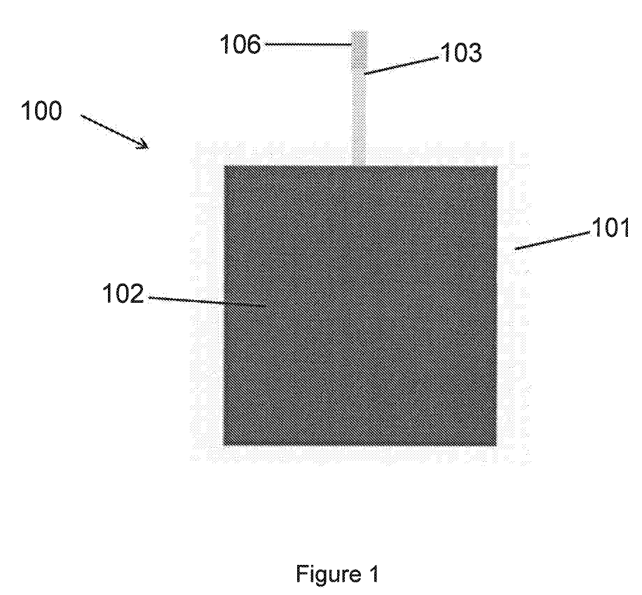

FIG. 1 shows a schematic representation of an example electrode according to the invention;

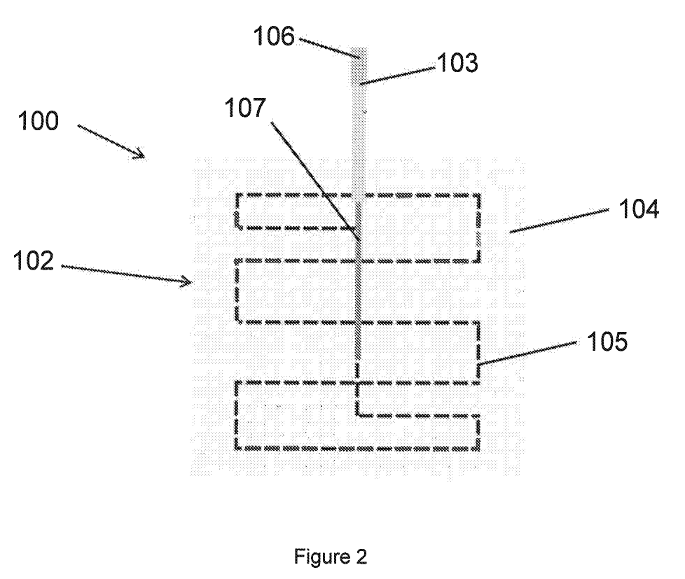

FIG. 2 shows another view of the electrode of FIG. 1;

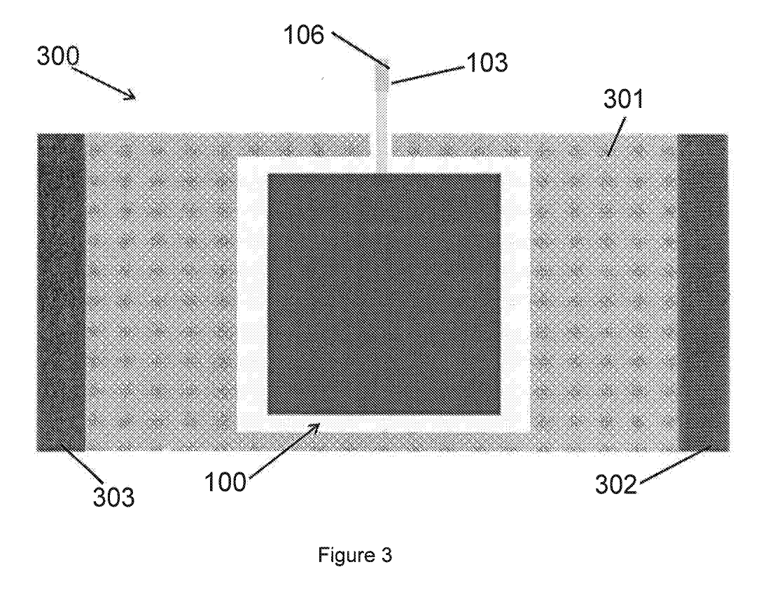

FIG. 3 shows a schematic representation of a garment incorporating the electrode of FIG. 1;

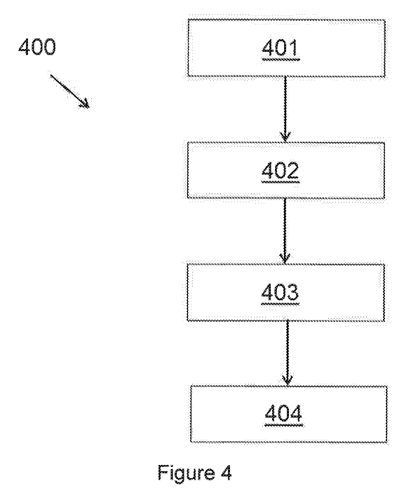

FIG. 4 is a flow chart of a method of producing an electrode; and

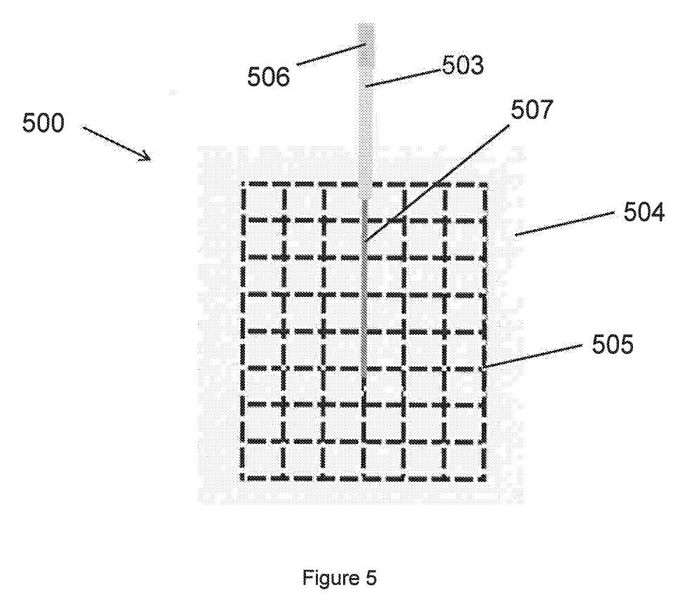

FIG. 5 shows an alternative arrangement of conductive yarn within an electrode according to the invention.

FIG. 1 shows an example of a wearable dry electrode 100 according to the invention. The electrode 100 comprises a conductive layer 101 and a contact layer 102 overlaying, and in electrical communication with, a portion of the conductive layer 101. The contact layer 102 is formed of a cured ink, the ink comprising a mixture of conductive carbon and silicone rubber. An external connector 103 is electrically connected to the conductive layer 101.

The contact layer 102 is adapted to form an electrical connection with the skin of a wearer, in order to detect electrical signals in the wearer's skin, or to apply electrical signals, such as small electrical currents, to the wearer's skin. In use, electrical signals pass from the contact layer 102 into the conductive layer 101, and from there into the external connector 103 and/or in the opposite direction, i.e. from the external connector 103 to the conductive layer 101 and from the conductive layer 101 to the contact layer 102. For example, the external connector 103 can be connected to an external device (not shown) for detecting electrical signals from the skin, such as a wearable medical device.

FIG. 2 shows the detail of the conductive layer 101 of electrode 100 without the contact layer 102. Conductive layer 101 comprises a textile 104. Conductive yarns 105 are integrated, for example sewn into, the textile 104. Conductive yarns 105 are adapted to carry electrical signals and may be made from a metallic material such as stainless steel, copper, silver, aluminium or an aluminium alloy: or metal-coated materials such as textile yarns coated with silver. External connector 103 is electrically connected to the conductive yarns 105. In the illustrated example, electrical connector 103 is a pigtail connector, comprising an electrical wire 107 connected to the conductive yarns 105 and a connector 106 connected to the electrical wire 107, the connector 106 being adapted to connect with an external device (not shown). For example, the external device may be plugged into the connector 106. It will be appreciated that a pigtail connector is simply an example of a suitable external connector 103.

In use, the conductive yarns 105 cannot come into contact with a user's skin, since the contact layer 102 completely overlays the conductive yarns (105) within the conductive layer 101.

Electrode 100 may be used for example as part of a wearable device such as a medical device, a healthcare device or a fitness device. Electrode 100 may provide a good contact with the skin for electrical signals to be detected in the skin and/or transmitted to the skin. Electrode 100 may therefore be used without requiring an extra interface layer, e.g. a gel, to be applied to increase the contact between the skin and the electrode. This may make electrode 100 easier to attach to a user's skin than conventional wet/hydrogel and dry electrodes, and more suited to long-term use than such conventional electrodes.

In particular examples, the electrode 100 may be integrated into a garment or item of clothing for a human or animal. One such example is shown in FIG. 3. FIG. 3 shows a garment 300, in this case an arm band. Garment 300 comprises an elasticated fabric 301. The electrode 100 is integrated into the fabric 301. The electrode 100 may for example be sewn into the fabric 301, adhered to the fabric 301 with an adhesive, or attached to the fabric 301 using hook and loop fasteners (e g. Velcro®).

Garment 300 further comprises a hook fastener 302 and a loop fastener 303. The hook fastener 302 is adapted to engage with the loop fastener 303. For example, the garment 300 may be wrapped around a user's arm, and secured by engaging the hook 302 and loop 303 fasteners.

Advantageously, the elasticated fabric 301 may ensure that the electrode 100 is kept in close contact with the skin of the wearer, whilst maintaining comfort for the wearer.

FIG. 4 shows a method of producing an electrode, such as electrode 100. In a first step 401, a conductive layer is provided. The conductive layer may be a conductive layer such as conductive layer 101, comprising conductive yarns integrated into a textile and electrically connected to an external connector.

At a second step 402, an ink is formed by mixing a silicone rubber with a conductive carbon powder. Graphene may be used as well as or instead of carbon powder. Alternatively, the ink or paste may be pre-mixed.

At step 403, the ink is printed or pasted onto the conductive layer. For example the ink may be stencil printed onto the conductive layer.

Finally at step 404, the ink may be cured, resulting in a dry electrode. Curing may for example comprise heating the ink to at least 60° C. and/or up to 150° C. for a period of at least 10 minutes and/or up to 60 minutes.

Alternatively, the contact layer may be provided as a preformed piece that is then attached, e.g. bonded, adhered and/or sewn, to the conductive layer.

Four particular ink formulations have been tested for use as electrodes. The compositions of these formulations are detailed in tables I and 2 below. The materials used in the compositions were:

Silicone rubber:

Viscolo 13A, Viscolo 13B: two parts silicone rubber purchased from TOMPS Online Limited, mix ratio of 1:1. Pot life: 40-50 minutes.

Ecoflex®, 00-10A, Ecoflex® 00-10B: two parts silicone rubber purchased from Smooth-on, mix ratio of 1:1. Pot life: 30 minutes. Ecoflex® 00-10A cures with a ‘tacky’ surface.

Thinner:

ESL T-402 thinner purchased from ESL ElectroScience.

Silicone Modifier:

Slacker® silicone modifier used to improve the tackiness of the cured Ecoflex silicone rubber, purchased from Smooth-on, Inc.

ENSACO 250G: conductive carbon powder supplied by Imerys Graphite & Carbon.

Each formulation comprised a silicone rubber (either Viscolo or EcoFlex®, as detailed above), mixed with conductive carbon. Formulations 3-4 further comprised a silicone modifier (Slacker®), to modify the tackiness of the silicone rubber. Both silicone rubbers (Viscolo and Ecoflex®) comprised two precursor formulations mixed together in a 1:1 ratio by weight to form the silicone rubber.

TABLE 1
Composition of ink formulation 1
Formulation 1

| Component | Proportions |
|---|---|
| Viscolo 13A | 50 |
| Viscolo 13B | 50 |
| Carbon | 15 |
| ESL T-402 (thinner) | 10 |

TABLE 2
Composition of ink formulations 2-4

| | Formulation 2 | Formulation 3 | Formulation 4 |
|---|---|---|---|
| Ecoflex 00-10 A | 50 | 50 | 25 |
| Ecoflex 00-10 B | 50 | 50 | 25 |
| Slacker (RTM) | 0 | 50 | 50 |
| Carbon | 15 | 22.5 | 15 |

By weight, formulation 1 comprised 15 parts carbon for every 100 parts Viscolo silicone rubber, and 10 parts thinner for every 100 parts silicone rubber. Formulation 1 did not include a silicone modifier.

By weight, formulation 2 comprised 15 parts carbon for every 100 parts Ecoflex® silicone rubber. Formulation 2 did not comprise a silicone modifier, or thinner.

By weight, formulation 3 comprised 22.5 parts carbon for every 100 parts Ecoflex® silicone rubber, and 50 parts Slacker® silicone modifier for every 100 parts silicone rubber.

By weight, formulation 4 comprised 30 parts carbon for every 100 parts Ecoflex® silicone rubber, and 100 parts silicone modifier for every 100 parts silicone rubber.

For each of the formulations, the components were mixed together to form a homogenous paste. The paste was stencil printed onto a conductive layer comprising stainless steel fibre conductive thread (purchased from Cool Components Ltd.), and cured at 80° C. for 30 minutes. Printing must occur soon after mixing, due to the limited pot-life of the silicone rubbers.

In formulation 1, the thinner solvent ESL T-402 was added to reduce the viscosity of the paste. However, the thinner solvent is not a required component of formulation 1. It was found that the electrode made with formulation 1 had no tackiness. The electrode based on formulation 2 had a light tackiness. The electrode based on formulation 3, which included the silicone modifier, provided a good tackiness. The additional silicone modifier added to formulation 4 made the paste difficult to cure, even with prolonged curing times. An electrode made with formulation 4 was cured at 120° C. for 30 minutes, resulting in a cured contact layer.

The electrodes made with formulations 1-4 were tested for a muscle simulation application—where the electrodes were used to apply an electrical signal to a muscle of a wearer. A MS2V2 (Odstock Medical Ltd) 2-channel electrical simulator was used. It was found that the formulation 2 electrode was more comfortable than the formulation 1 electrode. Due to the slight tackiness, the formulation 2 electrode offered a better skin-electrode contact than the formulation 1 electrode. The increased tackiness of the formulation 3 and 4 electrodes further increased the comfort, but less significantly than the difference between the formulation 1 and formulation 2 electrodes.

Further electrodes based on formulation 3 were also produced. These electrodes were cured at temperatures of 80° C., 100° C. and 120° C. respectively, with a cure time of 30 minutes. No noticeable effect on tackiness was found.

An electrode based on formulation 3 was washed 5 times with the washing condition of 30° C. 39 minutes and spin-speed of 600 rpm. The sample was dried at 80° C. for 10 minutes for each washing cycle. There is no visible damage and the tackiness is the same after 5 washes. The same results were observed when an electrode based on formulation 3 was washed 5 times with the washing condition of 30° C., 39 minutes and spin-speed of 1000 rpm. Accordingly, it will be appreciated that electrodes and garments according to the invention may be able to withstand repeated washing cycles. Consequently, electrodes and garments according to the invention may be relatively durable, long-lasting and/or reusable.

Thus electrodes produced with an ink according to formulations 2 or 3 may be particularly suited for use as wearable electrodes. They provide a dry electrode, which does not require a gel to increase the contact between the skin and electrode, and which is comfortable to wear and suitable for long-term use.

Electrodes with different arrangements of the conductive yarn within the textile were also produced FIG. 5 shows an alternative arrangement of conductive yarn 505 within a textile 504 of an electrode 500. The conductive yarn 505 is electrically connected to an external connector 503. The external connector 503 comprises a connector 506 adapted to connect with an external device (not shown) and an electrical wire 507 connected to the conductive yarns 505. Apart from the arrangement of the yarn 505 within the textile 504, electrode 500 was otherwise identical to the electrode 100 shown in FIGS. 1-2. It was found that for some wearers, the grid-like yarn arrangement shown in FIG. 5 resulted in a more comfortable electrode than the serpentine yarn arrangement shown in FIG. 2 if the contact layer was tacky. Conversely, it was found that for some wearers, the yarn arrangement shown in FIG. 2 resulted in a more comfortable electrode than the yarn arrangement shown in FIG. 5 if the contact layer was not tacky. Some wearers reported no noticeable comfort differences between the two yarn arrangements. It will be appreciated that the conductive yarns may be arranged in any suitable pattern, which may be determined at least in part by the intended use of a given electrode.

Conveniently, garments comprising electrodes according to the invention have been found to be flexible, conformable, easy to use, easy to maintain and unobtrusive. Accordingly, such garments may be suitable for use in long-term wearable applications, in particular long-term medical applications such as health monitoring, alleviating the symptoms of a medical condition, treating a medical condition or in rehabilitation.

Typically, any of the electrodes discussed above may be used to apply an electrical signal to the skin.

Potential uses for any of the electrodes discussed above include use as electrodes for functional electrical stimulation (FES) for stroke rehabilitation, or treatment of other neurological disorders—where the electrodes are used to apply an electrical signal to the skin. They may be used as electrodes used in transcutaneous electrical nerve stimulation (TENS) for pain relief. Example applications are pain relief for arthritis, back pain, neck pain.

By providing a dry electrode having good tackiness, skin contact is improved and consequently user comfort. Consequently, use of the invention may be particularly advantageous for muscle stimulation applications, as muscle stimulation typically requires a relatively high current density (mA/cm²) to be applied to the user's skin, in order to produce activation and movement in the case of rehabilitation. At such relatively high current density levels, use of a normal, dry electrode without a gel layer can lead to the user experiencing discomfort and even pain, due to the relatively poor skin contact. In contrast, the tackiness of the contact layer of the electrode of the present invention provides an improved interface between the electrode and the user's skin, which enables the relatively high current densities required for nerve or muscle stimulation to be applied to the user's skin without causing significant discomfort or pain. The tackiness of the contact layer of the electrode may be determined by the formulation of the ink or paste.

The electrodes may be used as electrodes for wearable biopotential monitoring systems such as ECG, EEG, EMG—where electrical signals in the skin are detected by the electrode.

Use of the present invention could provide significant savings in many applications.

For instance, the electrodes of the present invention may have potential to replace hydrogel electrodes, which are currently used as standard in Functional Electrical Stimulation (FES) for stroke rehabilitation. The typical usage lifetime for a pair of high quality hydrogel electrodes is around four weeks with a price of £4-5. Hence, the total cost per user is around £48-60/year. In contrast, the typical usage lifetime of the electrodes of the present invention may be considerably longer. Furthermore, by using the electrodes of the present invention rehabilitative treatment may be delivered more effectively and/or efficiently with less skilled intervention.

Advantageously, use of the electrodes of the present invention may address one or more of the following problems experienced by users of hydrogel electrodes for FES: difficulty in accurately positioning hydrogel electrodes, which can take 10-15 minutes per session according to our consultations with FES users; pain and hair removal when peeling the hydrogel electrodes from the skin, due to the stickiness of the hydrogel electrodes.

For patients recovering from a stroke, who have one impaired arm, there is an additional problem in that they typically cannot correctly use the hydrogel electrodes on their own; they need assistance from a career or healthcare professional. Consequently, such patients may not be able to perform their rehabilitation exercises independently. In contrast, the electrodes and garments of the present invention may be easier to use such that such patients are able to perform their rehabilitation exercises independently. Their being able to perform their rehabilitation exercises independently may reduce the burden on careers and healthcare providers and/or may increase the likelihood of a successful recovery.

Other neurological disorders that could be treated by FES using electrodes according to the invention include multiple sclerosis, spinal cord injury and Parkinson's disease.

A further advantage of the present invention is that the manufacture of the electrodes and garments according to the invention may be relatively straightforward and cost-effective. The manufacturing equipment required is either commercially available or can be made in a standard engineering workshop. Accordingly, it is envisaged that manufacture of the electrodes and garments would be readily scalable to larger volumes.

Other embodiments are intentionally within the scope of the invention as defined by the appended claims.

1. A wearable dry electrode comprising:
a conductive layer; and
a contact layer electrically connected to the conductive layer;
wherein the contact layer comprises carbon and a rubber.

2. The electrode of claim 1, wherein the contact layer comprises an ink or paste, the ink or paste comprising a mixture containing carbon and the rubber.

3. The electrode of claim 1, wherein the contact layer comprises, by weight, at least 5 and/or up to 40 parts carbon per 100 parts rubber.

4. The electrode of claim 1, wherein the contact layer further comprises a modifier for modifying the tackiness of the rubber.

5. The electrode of claim 4, wherein the contact layer comprises, by weight up to 110 parts modifier per 100 parts rubber.

6. The electrode of claim 1, wherein the contact layer further comprises a thinner solvent.

7. The electrode of claim 1, wherein the rubber comprises a silicone rubber.

8. The electrode of claim 1, wherein the conductive layer comprises at least one conductive yarn or conductive wire integrated into a textile of fabric.

9. The electrode of claim 1, wherein an external connector is electrically connected to the conductive layer.

10. A garment comprising:
a fabric; and
an electrode according to claim 1 attached to the fabric.

11. The garment of claim 10, wherein the electrode is integrated into the fabric or adhered to the fabric.

12. The garment of claim 10, wherein the fabric is an at least partially elasticated or stretchable fabric.

13. The garment of claim 10, wherein the garment comprises an arm band, chest band, hat, head band, sock, leg band, sleeve, t-shirt, shirt, leggings, tights shorts or body suit.

14. An ink or paste for forming the contact layer of the electrode according to claim 1, the ink or paste comprising a mixture of carbon and a rubber.

15. The ink or paste of claim 14, wherein the ink or paste comprises, by weight, between 5 and 40 parts carbon per 100 parts rubber.

16. The ink or paste of claim 14, wherein the ink or paste further comprises a modifier for modifying the tackiness of the rubber.

17. The ink or paste of claim 16, wherein the ink or paste comprises, by weight up to 110 parts modifier per 100 parts rubber.

18. The ink or paste of claim 14, wherein the ink or paste further comprises a thinner solvent.

19. The ink or paste of claim 14, wherein the rubber comprises a silicone rubber.

20-29. (canceled)
